(19) United States
(12) Patent Application Publication
(10) Pub. No.: US 2008/0138433 A1
(43) Pub. Date: Jun. 12, 2008

(54) VASODILATOR ELUTING BLOOD STORAGE AND ADMINISTRATION DEVICES WITH A SPECIFIC POLYPHOSPHAZENE COATING AND METHODS FOR THEIR MANUFACTURE AND USE

(75) Inventors: Olaf Fritz, Hirschhorn (DE); Ulf Fritz, Hirschhorn (DE); Roman Denk, Weidenstetten (DE); Teresa Wilson, Newnan, GA (US); Ralph E. Gaskins, Atlanta, GA (US)

Correspondence Address: SUTHERLAND ASBILL & BRENNAN LLP, 999 PEACHTREE STREET, N.E., ATLANTA, GA, 30309, US

(73) Assignee: CELONOVA BIOSCIENCES, INC., Newnan, GA (US)

(21) Appl. No.: 11/930,972

(22) Filed: Oct. 31, 2007

Related U.S. Application Data
(63) Continuation-in-part of application No. 11/023,928, filed on Dec. 28, 2004.

(30) Foreign Application Priority Data
Jul. 5, 2002 (DE) 10230190.5
Jul. 4, 2003 (EP) PCT/EP03/03197

(51) Int. Cl. A61K 35/14; C12M 1/00; A61L 27/34; A61L 27/54

(52) U.S. Cl. 424/529; 435/307.1; 514/772.3

(57) ABSTRACT

The present invention is directed to medical devices including blood storage and handling products that comprise a specific polyphosphazene and the capability of releasing nitric oxide or other smooth muscle relaxant compounds in vivo or into stored or transient flowing blood to achieve vascular dilatation, reduced adverse reactions, reduced thrombosis, reduced incidence of post-transfusion acute myocardial infarctions, and/or to improve blood storage capabilities.

### CROSS-REFERENCE TO RELATED APPLICATIONS

This application is a continuation-in-part of U.S. patent application Ser. No. 11/023,928, filed Dec. 28, 2004, which claims the benefit of priority of PCT Patent Application No. PCT/EP03/07197, filed Jul. 4, 2003 and German Patent Application No. DE10230190.5, filed Jul. 5, 2002, the entire disclosures of which are incorporated herein by reference.

### BACKGROUND OF THE INVENTION

The present invention is directed to blood storage and handling products that comprise a specific polyphosphazene and a capability of releasing nitric oxide or other smooth muscle relaxant compounds in vivo or into stored blood to achieve vascular dilatation, reduce adverse reactions, reduce thrombosis, reduce the incidence of post-transfusion acute myocardial infarction, and improve blood storage capabilities.

Nitric oxide (NO) is one of the few gaseous biological signaling molecules known. It is a key biological messenger, playing a role in a variety of biological processes. Nitric oxide, also known as the ‘endothelium-derived relaxing factor’, or ‘EDRF’, is biosynthesized from arginine and oxygen by various nitric oxide synthase MOS) enzymes and by reduction of inorganic nitrate. The endothelial cells that line blood vessels use nitric oxide to signal the surrounding smooth muscle to relax, thus dilating the artery and increasing blood flow. The production of nitric oxide is elevated in populations living at high-altitudes, which helps these people avoid hypoxia. Effects include blood vessel dilatation, and neurotransmission. Nitroglycerin and amyl nitrite serve as vasodilators because they are converted to nitric oxide in the body.

Phosphodiesterase type 5 inhibitors, often shortened to PDE5 inhibitors are a class of drugs used to block the degradative action of phosphodiesterase type 5 on cyclic GMP in the smooth muscle cells lining blood vessels. NO activates the enzyme guanylate cyclase which results in increased levels of cyclic guanosine monophosphate (cGMP), leading to smooth muscle relaxation in blood vessels. PDE5 inhibitors inhibit the degradation of cGMP by phosphodiesterase type 5 (PDE5).

Nitric oxide is also generated by macrophages and neutrophils as part of the human immune response. Nitric oxide is toxic to bacteria and other human pathogens. In response, however, many bacterial pathogens have evolved mechanisms for nitric oxide resistance.

A biologically important reaction of nitric oxide is S-nitrosylation, the conversion of thiol groups, including cysteine residues in proteins, to form S-nitrosothiols (RSNOs). S-Nitrosylation is a mechanism for dynamic, post-translational regulation of most or all major classes of protein.

Nitroglycerine or glyceryl trinitrate (GTN) has been used to treat angina and heart failure since at least 1880. Despite this, the mechanism of nitric oxide (NO) generation from GTN and the metabolic consequences of this bioactivation are still not entirely understood.

GTN is a pro-drug which must first be denitrated to produce the active metabolite NO. Nitrates which undergo denitration within the body to produce NO are called nitrovasodilators and their denitration occurs via a variety of mechanisms. The mechanism by which nitrates produce NO is widely disputed. Some believe that nitrates produce NO by reacting with sulfhydryl groups, while others believe that enzymes such as glutathione S-transferases, cytochrome P450 (CYP), and xanthine oxidoreductase are the primary source of GTN bioactivation. In recent years a great deal of evidence has been produced which supports the belief that clinically relevant denitration of GTN to produce 1,2-glyceryl dinitrate (GDN) and NO is catalyzed by mitochondrial aldehyde dehydrogenase (mtALDH). NO is a potent activator of guanylyl cyclase (GC) by heme-dependent mechanisms; this activation results in cGMP formation from guanosine triphosphate (GTP). Thus, NO increases the level of cGMP within the cell.

GTP is more useful in preventing angina attacks than reversing them once they have commenced. Patches of glyceryl trinitrate with long activity duration are commercially available. It may also be given as a sublingual dose in the form of a tablet placed under the tongue or a spray into the mouth for the treatment of an angina attack.

Long acting Nitrates can be more useful as they are generally more effective and stable in the short term. GTP is also used to help provoke a vasovagal syncope attack while having a tilt table test which will then give more accurate results.

A study published in the Proceedings of the National Academy of Science, Oct. 11, 2007, “S-nitrosohemoglobin deficiency: A mechanism for loss of physiological activity in banked blood” by Reynolds J D, et al. suggests that 70% of Nitric Oxide (NO) is depleted from banked blood by the end of its 42-day shelf life. In that study, 24,000 people with acute coronary syndrome (ACS) showed that those who got a transfusion had a 25% chance of acute myocardial infarction (AMI) and 8% died. Those without a transfusion had an 8% chance of AMI and 3% chance of death. Previous trials have shown that heart disease patients, for example, who receive a blood transfusion to help restore oxygen to deprived tissues, have a 25% chance of having a heart attack and an 8% chance of dying within 30 days; similar patients who do not get transfused have an 8% chance of a cardiac event and a 3% chance of death. It has been hypothesized that, without adequate nitrous oxide in the blood, red blood cells cannot make their way into tiny blood vessels; rather, they pile up in narrow lumens, obstructing blood flow and adversely affecting organ function.

Febrile non-hemolytic transfusion reaction is the most common adverse reaction to a blood transfusion. Symptoms include fever and dyspnea 1 to 6 hours after receiving the transfusion. Such reactions are clinically benign, causing no lasting side effects or problems, but are unpleasant via a blood transfusion is estimated, as of 2006, at 1 per 2 million units transfused. Bacterial infection is a much more common problem.

Blood products can provide an excellent medium for bacterial growth, and can become contaminated after collection while they are being stored. The risk is highest with platelet transfusion, since platelets must be stored near room temperature and cannot be refrigerated. The risk of severe bacterial infection and sepsis is estimated (as of 2001) at about 1 in 50,000 platelet transfusions, and 1 in 500,000 red blood cell transfusions.

Acute hemolytic reaction is a medical emergency resulting from rapid destruction (hemolysis) of the donor red blood cells by host antibodies. The most common cause is clerical error (i.e. the wrong unit of blood being given to the wrong patient). The symptoms are fever and chills, sometimes with back pain and pink or red urine (hemoglobinuria). The major complication is that hemoglobin released by the destruction of red blood cells can cause acute renal failure.

An anaphylactic (or severe allergic) reaction can occur at a rate of 1 per 30,000-50,000 transfusions. These reactions are most common in people with selective IgA deficiency (although IgA deficiency is often asymptomatic, and people may not know they have it until an anaphylactic reaction occurs). An anaphylactic reaction is a medical emergency, requiring prompt treatment, and may be life-threatening.

Transfusion-associated acute lung injury (TRALI) is a syndrome of acute respiratory distress, often associated with fever, non-cardiogenic pulmonary edema, and hypotension. It may occur as often as 1 in 2000 transfusions. Symptoms can range from mild to life-threatening, but most patients recover fully within 96 hours, and the mortality rate from this condition is less than 10%.

Patients with impaired cardiac function (such as congestive heart failure) can become volume-overloaded as a result of blood transfusion, leading to edema, dyspnea (shortness of breath), and orthopnea (shortness of breath while lying flat). This is sometimes called TACO, or Transfusion Associated Circulatory Overload.

Each transfused unit of red blood cells contains approximately 250 mg of elemental iron. Since elimination pathways for iron are limited, a person receiving numerous red blood cell transfusions can develop iron overload, which can in turn damage the liver, heart, kidneys, and pancreas. The threshold at which iron overload becomes significant is somewhat unclear, but is likely around 12-20 units of red blood cells transfused.

Transfusion-associated graft-vs-host disease (GVHD) refers to an immune attack by transfused cells against the recipient. This is a common complication of stem cell transplantation, but an exceedingly rare complication of blood transfusion. It occurs only in severely immunosuppressed patients, primarily those with congenital immune deficiencies or hematologic malignancies who are receiving intensive chemotherapy. When GVHD occurs in association with blood transfusion, it is almost uniformly fatal. Transfusion-associated GVHD can be prevented by irradiating the blood products prior to transfusion.

Thus, there exists in the art a need for medical devices capable of eluting nitric oxide in the areas of vascular devices to maintain vasodilatation for periods of time, and in blood banking and the handling of blood products.

Blood banking containers and accessories used in the handling of blood are among the items that could benefit from an inherent ability to increase and maintain nitric oxide levels within the contained and processed blood and blood products. Blood bags are commercially available for the collection, storage, and processing of blood and blood products, such as packed red cells, plasma, platelets, and cryoprecipitate.

Most currently-available blood bags are made of a polyvinyl chloride (PVC) formulation which includes, as a plasticizer, di-2-ethylhexylphthalate. Such a plasticizer is absolutely necessary for polyvinyl chloride formulations, since polyvinyl chloride itself is not a suitable, flexible plastic material for use in containers. Such blood bags have served extremely well in the storage and processing of blood and blood components, exhibiting a high survival rate with low plasma hemoglobin content after, for example, 21 days of storage at about 4° C.

Blood transfusion became safe, dependable and convenient and a result of three important developments—the landmark discovery of blood groups, the safety and effectiveness of citrate for intra venous administration, and the development of the anticoagulant solution ACD. Modern blood banking, however, was initiated with the pioneering work of development of PVC bags for blood collection and storage in 1947.

Plasticized PVC containers possess a number of advantages which makes it the material of choice for medical and more particularly for blood contact applications. PVC containers' more important features include: ability to be welded together which enables the production of leak-free products and offers infinite design possibilities, steam sterilizability even at 121° C., favorable cost/performance ratio, and low bulk density offering low storage and distribution costs. The plasticizers used in the compounding of PVC are mainly responsible for achieving desirable characteristics for medical applications such as low toxicity, transparency, flexibility, strengths, elongation, stability at low and high temperatures, permeability to water, oxygen and carbon dioxide in the desired range. While a wide range of plasticizers are available for food contact and most medical applications, the choices for blood contact applications are very limited. The principal plasticizer for blood containers and related applications used is di, (2-ethyl hexyl) phthalate (DEHP) which offers the benefits of overall performance, ready availability at high purity and cost effectiveness.

DEHP is not covalently bonded to PVC and so could migrate out of the plastic. This is particularly so in the presence of solubilizing lipids, lipoproteins and albumin. Jaegar and Rubin (1970) reported the leaching of DEHP into stored human blood. Several studies conducted subsequently demonstrated the extractability of DEHP from PVC blood bag into whole blood, platelet concentrates and plasma during storage. Concerns have been expressed about the adverse effects of DEHP leached into blood and blood products and extensive studies have been made to assess every facet of the problems reported including excellent reviews. PVC materials plasticized with DEHP have been used in patient health care for over 50 years and there are over 3000 published papers discussing its potential toxicological hazards. A recent report by Dr Everett Koop (June 1999) former Surgeon General and Chairman of an expert panel convened by the American Council on Science and Health, sums up the present position succinctly “DEHP in medical devices is not harmful to even highly exposed people, those who undergo certain medical procedures such as regular hemodialysis, or extra corporeal membrane oxygenation. The panel concluded that “DEHP imparts a variety of important physical characteristics that are critical to the function of medical devices and eliminating DEHP in these products could cause harm.”

It is now well established that platelets could be stored with reasonable post transfusion recovery and survival for up to 72 hours in DEHP plasticized blood bags. In an earlier study, it was demonstrated that thin walled DEHP plasticized PVC containers behaved better indicating thereby that higher oxygen and carbon dioxide permeabilities were desirable.

The effect of DEHP plasticizer on stored platelets was studied by Racz and Baroti (1995). They found that:
Platelet aggregation was the only parameter that was slightly inhibited in DEHP plasticized bags indicating that the presence of DEHP had no harmful effect during storage.
Platelets from 400 ml donor blood could be stored in DEHP plasticized bags meant for storage of platelets from 450 ml of blood without deterioration. Such platelets were not inferior to platelet concentrates stored for 5 days in PL 732 (Polyolefin) or PL 1240 (BTHC) containers.

Grode, et al studied the storage of platelet concentrates in PVC bags plasticized with tri-octyl, tri-mellitate (TOTM) plasticizer and showed that such bags possessed sufficient gas permeability to be suitable for extended storage of platelets for at least 5 days. They also found a 30 fold reduction in plasticizer accumulation in platelet concentrates as compared to DEHP plasticized bags. Studies made subsequently confirm that such bags may be used for storing platelet concentrates for at least 5 days at 22oC. While TOTM plasticized containers were found satisfactory for storage of most platelet concentrates, it may be desirable to use more permeable containers if platelet yields are routinely very high. A distinct advantage of TOTM is its low migration and volatility characteristics.

Blood bags made with n-butyrul, tri n-hyxyl citrate (BTHC) plasticizers have been shown to be effective for storing platelets and their behavior is similar to TOTM plasticized bags. Measurements of pH, pO2, pCO2, glucose, lactate, ATP, total adenine nucleotide, lactate dehydrogenase and platelet factor-4 (pF4) showed similar results for BTHC and TOTM plasticized bags during five day storage of platelets. Results of in vivo studies were similar. However, studies have suggested that while BTHC and polyolefin containers ensure sufficient oxygenation to maintain aerobic metabolism, their carbon dioxide permeability is too high and allows too much escape of the CO2 gas.

Modified polyolefins are suitable for the storage of platelets for up to seven days because of their higher gas permeability. However, aberrant morphology has been observed after 2-3 days. Such modified polyolefins are reported to be free of plasticizers, but may contain antioxidants to prevent oxidative deterioration. Polyolefin materials have also been shown to give rise to leaching. Thus polypropylene releases many low molecular weight oligomers while polyethylene releases higher molecular weight oligomers.

PVC blood bags are also available incorporating a less leachable phthalate ester-di, n-decyl phthalate (DnDP) which has better gas permeabilities. The leachability of this plasticizer into plasma is 1/80th that of DEHP. The LD50 values of DnDP is approximately ten times that of DEHP. This makes DnDP appear more than 800 times safer than DEHP. The new bag has been shown to be suitable for the five day storage of platelet concentrates.

Adverse transfusion reactions are encountered in 2%-10% of patients receiving transfusions; such reactions may range from infectious blood-borne diseases or allergic reactions to death in some individuals patients. Repeatedly, medical studies have been reported, showing disturbing increased rates in heart disease and death in patients who previously received transfusions.

Therefore, it would be desirable to improve the risk of heart attack and death from transfusion by replacing nitric oxide in stored blood.

It would also be desirable to therapeutically increase the nitrous oxide content in blood in vivo in anatomic areas for treatment for diseases or pathologic conditions in which localized or systemic vasodilatation is compromised.

It would further be desirable to be able to therapeutically increase the nitrous oxide content in blood during in vivo procedures such as autotransfusion in which blood is being removed from a patient's body, circulated through pumping or filtering devices, and returned to the patient.

According to the present invention, polyvinyl chloride or other plastic blood bags, administration tubing, and other blood-handling or storage devices may be formulated to provide desirable physical characteristics, along with a coating capable of both reducing the plasma concentration of extractable plasticizer and eluting nitrous oxide to provide decreased transfusion-related morbidity and mortality.

### BRIEF SUMMARY OF THE INVENTION

The invention includes a coating for medical devices for use blood storage, handling, and administration in therapeutic settings where it is desirable to have such devices release nitric oxide or other smooth muscle relaxant drugs into blood.

The medical devices of the present invention further comprise poly[bis(trifluoroethoxy)phosphazene] and/or a derivative thereof and one or more smooth muscle relaxant active agents. Poly[(bistrifluorethoxy)phosphazene] has antibacterial and anti-inflammatory properties and inhibits the accumulation of thrombocytes.

Further described herein is a method of delivering an active agent capable of eluting nitric oxide or other smooth muscle relaxants from within a specific polyphosphazene coating into an anatomic area or a container space is therapeutically desirable.

### BRIEF DESCRIPTION OF THE SEVERAL VIEWS OF THE DRAWINGS

The foregoing summary, as well as the following detailed description of the invention, will be better understood when read in conjunction with the appended drawings. For the purpose of illustrating the invention, there are shown in the drawings embodiments that are presently preferred. It should be understood, however, that the invention is not limited to the precise arrangements and instrumentalities shown.

In the drawings:

FIG. 1A shows a surface of a film of the present invention.

FIG. 1B shows a cross section of the film of the present invention from FIG. 1A.

FIG. 2A shows a blood bag assembly comprising a blood bag and blood administration tubing.

FIG. 2B shows a cross section of a blood administration tubing of the present invention.

FIG. 2C shows a cross section of a wall of a blood bag of the present invention.

### DETAILED DESCRIPTION OF THE INVENTION

The present invention may be understood more readily by reference to the following detailed description of the preferred embodiments of the invention and the examples included herein. However, before the preferred embodiments of the devices and methods according to the present invention are disclosed and described, it is to be understood that this invention is not limited to the exemplary embodiments described within this disclosure, and the numerous modifications and variations therein that will be apparent to those skilled in the art remain within the scope of the invention disclosed herein. It is also to be understood that the terminology used herein is for the purpose of describing specific embodiments only and is not intended to be limiting.

Unless otherwise noted, the terms used herein are to be understood according to conventional usage by those of ordinary skill in the relevant art. In addition to the definitions of terms provided below, it is to be understood that as used in the specification and in the claims, “a” or “an” can mean one or more, depending upon the context in which it is used.

Described herein are medical devices for the storage, handling, and administration of blood or blood products, said devices comprising poly[bis(trifluoroethoxy)phosphazene] and/or a derivative thereof and one or smooth muscle relaxant active agents capable of in vivo release into blood or blood products stored or administered using said devices.

Further described herein are methods for the manufacture and use of medical devices comprising poly[bis(trifluoroethoxy)phosphazene] and/or a derivative thereof and one or more nitrogen compounds or other smooth muscle relaxant active agents capable of release during storage of biological or pharmaceutical containment or administration therein, or in vivo release into the tissues or organs of a mammalian patient upon implantation, deployment, or use of said devices.

In certain embodiments of the present invention, medical devices for the storage, handling, and administration of blood or blood products are provided with a polymeric coating comprising poly[bis(trifluoroethoxy)phosphazene] and/or a derivative thereof releasably bonded to compounds capable of producing nitric oxide or other bioactive nitrogen compounds upon release from the polymer.

The present invention further includes methods for the manufacture and use of medical devices for the storage, handling, and administration of blood or blood products comprising a polymeric coating comprising poly[bis(trifluoroethoxy)phosphazene] and/or a derivative thereof releasably bonded to compounds capable of producing nitric oxide or other bioactive nitrogen compounds upon release from the polymer.

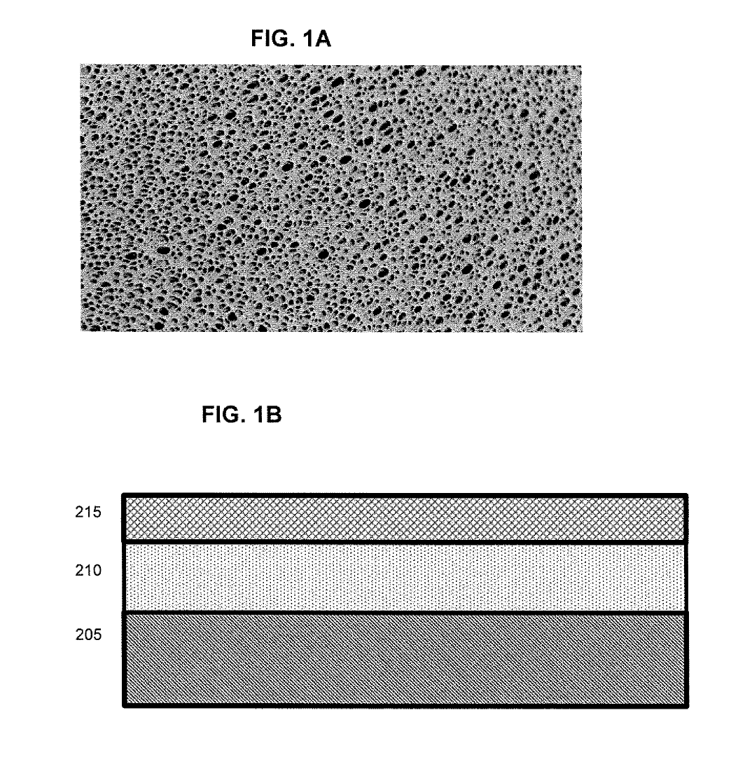

FIG. 1A shows an exemplary surface view of a film surface of a medical device of the present invention. A cross section through such a film surface is shown in FIG. 1B, where a substrate medical device surface 205 is coated with the poly[bis(trifluoroethoxy)phosphazene] polymer represented by formula (I) in an exterior polymeric coat 215, with an intermediate smooth muscle relaxant coating 210 sandwiched between the substrate 205 and the polymeric coat 215.

The intermediate smooth muscle relaxant coating 210 as shown in FIG. 1B may be any nitrogen compound capable of in vivo breakdown to nitric oxide or other vasoactive nitrite or nitrate compounds. In alternate embodiments of the present invention, the intermediate smooth muscle relaxant coating 210 may be a non-nitrogen based smooth muscle relaxant agent. In the exemplary FIG. 1B section, the intermediate smooth muscle relaxant coating 210 is shown as a separate layer, adherent to the substrate 205 and covalently bonded or otherwise adherent to the exterior polymeric coat 215. In still other embodiments of the present invention, smooth muscle relaxant agent may be integrated into the exterior polymeric coat 215.

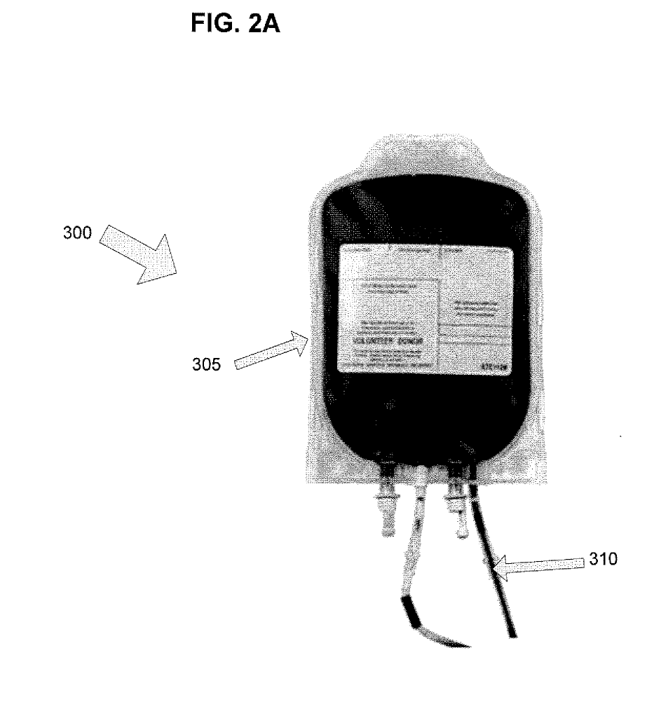

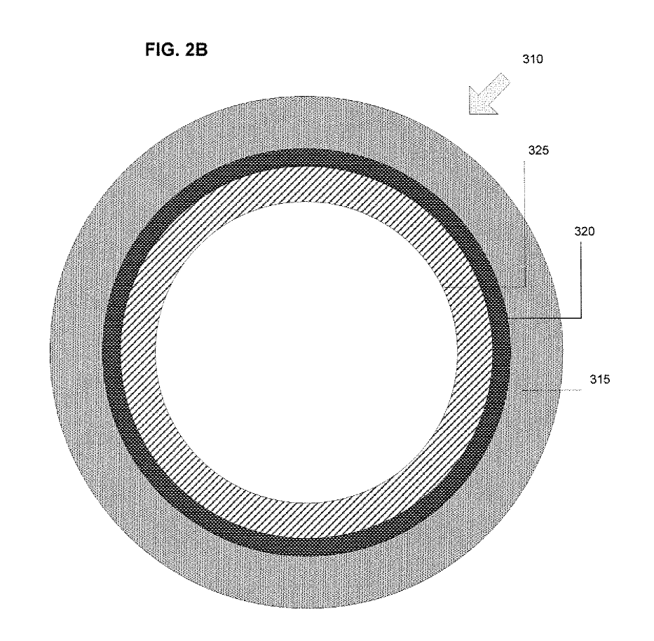

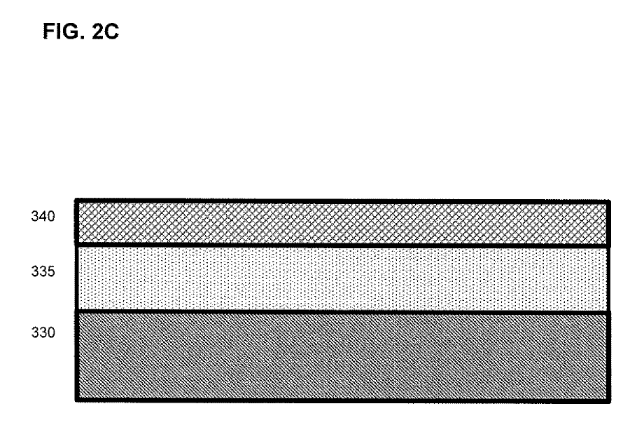

FIG. 2A shows an exemplary plastic blood bag assembly 300, comprising a blood bag 305 and a blood administration tubing 310. Blood banks commonly use such blood bag assemblies 300 to collect, process, store, and administer blood or blood product transfusions. A blood bag assembly 300 of the present invention externally may appear the same as a conventional product, but further comprises an inner polymeric coating comprising poly[bis(trifluoroethoxy)phosphazene] and/or a derivative thereof releasably bonded to compounds capable of producing nitric oxide or other bioactive nitrogen compounds upon release from the polymer.

An exemplary blood bag assembly 300 of the present invention provides a gradual release of nitric oxide into the blood or blood product(s) contained within said blood bag assembly 300, thus improving product shelf life and reducing the incidence and severity of transfusion reactions associated with the administration of transfusions of the contained blood or blood product(s) into patients.

FIG. 2B shows a cross-sectional view through a representative section of an exemplary blood administration tubing 310 of the present invention. As shown in FIG. 2B, the blood administration tubing 310 comprises an outer tubing wall 315, an inner polymeric coating 325, and an intermediate nitric oxide eluting layer 320.

In use, blood or blood products either contained within blood administration tubing 310 or transient therethrough receives nitric oxide, produced by gradual chemical breakdown of the an intermediate nitric oxide eluting layer 320. The inner polymeric coating 325, comprising poly[bis(trifluoroethoxy)phosphazene] and/or a derivative thereof, imparts antithrombotic, anti-inflammatory, and lubricious qualities to the inner wall of the blood administration tubing 310, thus reducing blood cell or platelet injuries or clot formation during containment or administration.

The intermediate nitric oxide eluting layer 320 as shown in FIG. 2B may be any nitrogen compound capable of in vivo breakdown to nitric oxide or other nitrite or nitrate compounds. In the exemplary FIG. 2B section, the intermediate nitric oxide eluting layer 320 is shown as a separate layer, adherent to the inner layer of the tubing wall 315 and covalently bonded or otherwise adherent to the inner polymeric coating 325. In still other embodiments of the present invention, nitric oxide-eluting compounds may be integrated into the inner polymeric coating 325.

FIG. 2C shows a cross-sectional view through a representative section of an exemplary blood bag 305 of the present invention, comprising an outer blood bag wall 330, an inner polymeric coating 340, and an intermediate nitric oxide eluting layer 335.

In use, as with the exemplary blood administration tubing 310, blood or blood products either contained within a blood bag 305 of the present invention receives nitric oxide, produced by gradual chemical breakdown of the an intermediate nitric oxide eluting layer 335. The inner polymeric coating 340, again comprising poly[bis(trifluoroethoxy)phosphazene] and/or a derivative thereof, imparts antithrombotic, anti-inflammatory, and lubricious qualities to the inner wall of the blood bag 305, thus reducing blood cell or platelet injuries or clot formation during containment or administration.

The intermediate nitric oxide eluting layer 335 as shown in FIG. 2C may be any nitrogen compound capable of breakdown to nitric oxide or other nitrite or nitrate compounds. In the exemplary FIG. 2C section, the intermediate nitric oxide eluting layer 335 is shown as a separate layer, adherent to the inner layer of the blood bag wall 330 and covalently bonded or otherwise adherent to the inner polymeric coating 340. In still other embodiments of the present invention, nitric oxide-eluting compounds may be integrated into the inner polymeric coating 340.

The intermediate nitric oxide eluting layer 335 may further be releasable from its chemical bond to the inner polymeric coating 340 or the inner layer of the blood bag wall 330 in a time release manner, and/or such release may be accelerated or activated by the administration of releasing agents such as pH-altering agents, or by the administration of energy in the forms of thermal or electromagnetic radiation energy at desired times.

As described herein, the polymer poly[bis(2,2,2-trifluoroethoxy)phosphazene] or derivatives thereof have chemical and biological qualities that distinguish this polymer from other know polymers in general, and from other know polyphosphazenes in particular, In one aspect of this invention, the polyphosphazene is poly[bis(2,2,2-trifluoroethoxy)phosphazene] or derivatives thereof, such as other alkoxide, halogenated alkoxide, or fluorinated alkoxide substituted analogs thereof. The preferred poly[bis(trifluoroethoxy)phosphazene] polymer is made up of repeating monomers represented by the formula (I) shown below:

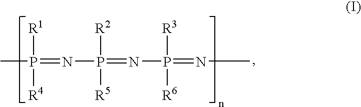

wherein R¹ to R⁶ are all trifluoroethoxy (OCH₂CF₃) groups, and wherein n may vary from at least about 40 to about 100,000, as disclosed herein. Alternatively, one may use derivatives of this polymer in the present invention. The term “derivative” or “derivatives” is meant to refer to polymers made up of monomers having the structure of formula I but where one or more of the R¹ to R⁶ functional group(s) is replaced by a different functional group(s), such as an unsubstituted alkoxide, a halogenated alkoxide, a fluorinated alkoxide, or any combination thereof, or where one or more of the R¹ to R⁶ is replaced by any of the other functional group(s) disclosed herein, but where the biological inertness of the polymer is not substantially altered.

In one aspect of the polyphosphazene of formula (I) illustrated above, for example, at least one of the substituents R¹ to R⁶ can be an unsubstituted alkoxy substituent, such as methoxy (OCH₃), ethoxy (OCH₂CH₃) or n-propoxy (OCH₂CH₂CH₃). In another aspect, for example, at least one of the substituents R¹ to R⁶ is an alkoxy group substituted with at least one fluorine atom. Examples of useful fluorine-substituted alkoxy groups R¹ to R⁶ include, but are not limited to OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, and the like. Thus, while trifluoroethoxy (OCH₂CF₃) groups are preferred, these further exemplary functional groups also may be used alone, in combination with trifluoroethoxy, or in combination with each other. In one aspect, examples of especially useful fluorinated alkoxide functional groups that may be used include, but are not limited to, 2,2,3,3,3-pentafluoropropyloxy (OCH₂CF₂CF₃), 2,2,2,2′,2′,2′-hexafluoroisopropyloxy (OCH(CF₃)₂), 2,2,3,3,4,4,4-heptafluorobutyloxy (OCH₂CF₂CF₂CF₃), 3,3,4,4,5,5,6,6,7,7,8,8,8-tridecafluorooctyloxy (OCH₂(CF₂)₇CF₃), 2,2,3,3,-tetrafluoropropyloxy (OCH₂CF₂CHF₂), 2,2,3,3,4,4-hexafluorobutyloxy (OCH₂CF₂CF₂CHF₂), 3,3,4,4,5,5,6,6,7,7,8,8-dodecafluorooctyloxy (OCH₂(CF₂)₇CHF₂), and the like, including combinations thereof.

Further, in some embodiments, 1% or less of the R¹ to R⁶ groups may be alkenoxy groups, a feature that may assist in crosslinking to provide a more elastomeric phosphazene polymer. In this aspect, alkenoxy groups include, but are not limited to, OCH₂CH═CH₂, OCH₂CH₂CH═CH₂, allylphenoxy groups, and the like, including combinations thereof. Also in formula (I) illustrated herein, the residues R¹ to R⁶ are each independently variable and therefore can be the same or different.

By indicating that n can be as large as ∞ in formula I, it is intended to specify values of n that encompass polyphosphazene polymers that can have an average molecular weight of up to about 75 million Daltons. For example, in one aspect, n can vary from at least about 40 to about 100,000. In another aspect, by indicating that n can be as large as so in formula I, it is intended to specify values of n from about 4,000 to about 50,000, more preferably, n is about 7,000 to about 40,000 and most preferably n is about 13,000 to about 30,000.

In another aspect of this invention, the polymer used to prepare the polymers disclosed herein has a molecular weight based on the above formula, which can be a molecular weight of at least about 70,000 g/mol, more preferably at least about 1,000,000 g/mol, and still more preferably a molecular weight of at least about 3×10⁶ g/mol to about 20×10⁶ g/mol. Most preferred are polymers having molecular weights of at least about 10,000,000 g/mol.

In a further aspect of the polyphosphazene formula (I) illustrated herein, n is 2 to ∞, and R¹ to R⁶ are groups which are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof or heteroaryl comprising one or more heteroatoms selected from nitrogen, oxygen, sulfur, phosphorus, or a combination thereof, In this aspect of formula (I), the pendant side groups or moieties (also termed “residues”) R¹ to R⁶ are each independently variable and therefore can be the same or different. Further, R¹ to R⁶ can be substituted or unsubstituted. The alkyl groups or moieties within the alkoxy, alkylsulphonyl, dialkylamino, and other alkyl-containing groups can be, for example, straight or branched chain alkyl groups having from 1 to 20 carbon atoms, typically from 1 to 12 carbon atoms, it being possible for the alkyl groups to be further substituted, for example, by at least one halogen atom, such as a fluorine atom or other functional group such as those noted for the R¹ to R⁶ groups above. By specifying alkyl groups such as propyl or butyl, it is intended to encompass any isomer of the particular alkyl group.

In one aspect, examples of alkoxy groups include, but are not limited to, methoxy, ethoxy, propoxy, and butoxy groups, and the like, which can also be further substituted. For example the alkoxy group can be substituted by at least one fluorine atom, with 2,2,2-trifluoroethoxy constituting a useful alkoxy group. In another aspect, one or more of the alkoxy groups contains at least one fluorine atom. Further, the alkoxy group can contain at least two fluorine atoms or the alkoxy group can contain three fluorine atoms. For example, the polyphosphazene that is combined with the silicone can be poly[bis(2,2,2-trifluoroethoxy)phosphazene]. Alkoxy groups of the polymer can also be combinations of the aforementioned embodiments wherein one or more fluorine atoms are present on the polyphosphazene in combination with other groups or atoms.

Examples of alkylsulphonyl substituents include, but are not limited to, methylsulphonyl, ethylsulphonyl, propylsulphonyl, and butylsulphonyl groups. Examples of dialkylamino substituents include, but are not limited to, dimethyl-, diethyl-, dipropyl-, and dibutylamino groups. Again, by specifying alkyl groups such as propyl or butyl, it is intended to encompass any isomer of the particular alkyl group.

Exemplary aryloxy groups include, for example, compounds having one or more aromatic ring systems having at least one oxygen atom, non-oxygenated atom, and/or rings having alkoxy substituents, it being possible for the aryl group to be substituted for example by at least one alkyl or alkoxy substituent defined above. Examples of aryloxy groups include, but are not limited to, phenoxy and naphthoxy groups, and derivatives thereof including, for example, substituted phenoxy and naphthoxy groups.

The heterocycloalkyl group can be, for example, a ring system which contains from 3 to 10 atoms, at least one ring atom being a nitrogen, oxygen, sulfur, phosphorus, or any combination of these heteroatoms. The heterocycloalkyl group can be substituted, for example, by at least one alkyl or alkoxy substituent as defined above. Examples of heterocycloalkyl groups include, but are not limited to, piperidinyl, piperazinyl, pyrrolidinyl, and morpholinyl groups, and substituted analogs thereof.

The heteroaryl group can be, for example, a compound having one or more aromatic ring systems, at least one ring atom being a nitrogen, an oxygen, a sulfur, a phosphorus, or any combination of these heteroatoms. The heteroaryl group can be substituted for example by at least one alkyl or alkoxy substituent defined above. Examples of heteroaryl groups include, but are not limited to, imidazolyl, thiophene, furane, oxazolyl, pyrrolyl, pyridinyl, pyridinoyl, isoquinolinyl, and quinolinyl groups, and derivatives thereof, such as substituted groups.

As disclosed herein, smooth muscle relaxant active agents or compounds capable of producing nitric oxide or other bioactive nitrogen compounds upon release of the present invention further comprise diazeniumdiolates, sodium nitroprusside, molsidomine, nitrate esters, the S-nitrosothiol family, L-arginine, nitric oxide-nucleophile complexes, glyceryl trinitrate, nitric oxide-primary amine complexes, and related compounds, esters, amines, or other compositions thereof. Smooth muscle relaxant active agents or compounds capable of producing nitric oxide or other bioactive nitrogen compounds upon release of the present invention may further comprise any other inorganic or organic composition capable of forming nitric oxide upon chemical degradation.

In certain preferred embodiments of the present invention, diazeniumdiolates are incorporated into blood-insoluble polyphosphazene polymers that generate molecular NO at their surfaces. In other preferred embodiments of the present invention, diazeniumdiolates may be applied to a substrate surface of a medical device as an intermediate coating, which is then coated with the preferred poly[bis(trifluoroethoxy)phosphazene] polymer of the present invention. In yet other preferred embodiments of the present invention, a substrate inner surface of a blood storage or handling device may receive a first coating with the preferred poly[bis(trifluoroethoxy)phosphazene] polymer of the present invention, followed by an intermediate coating of diazeniumdiolates, followed by a second coating of the poly[bis(trifluoroethoxy)phosphazene] polymer as described herein. In such embodiments with a first and second coating of the poly[bis(trifluoroethoxy)phosphazene] polymer, the first and second coatings may each be bioabsorbable or non-bioabsorbable.

Diazeniumdiolates are now available with a range of half-lives for spontaneous NO release. The ability of the diazeniumdiolates to generate copious NO at rates that vary widely is largely independent of metabolic or medium effects.

Other preferred embodiments of the present invention may use other nitric oxide-eluting or other smooth muscle relaxant compounds, including, but not limited to sodium nitroprusside, molsidomine, nitrate esters, the S-nitrosothiol family, L-arginine, nitric oxide-nucleophile complexes, glyceryl trinitrate, nitric oxide-primary amine complexes, and related compounds. In such various embodiments of the present invention, the nitric oxide-eluting or other smooth muscle relaxant compounds may be incorporated into non-bioabsorbable polyphosphazene polymers that generate molecular NO at their surfaces. In other preferred embodiments of the present invention, nitric oxide-eluting or other smooth muscle relaxant compounds may be applied to a substrate inner surface of a blood storage or handling device as an intermediate coating, which is then coated with the preferred poly[bis(trifluoroethoxy)phosphazene] polymer of the present invention. In yet other preferred embodiments of the present invention, a substrate surface of a medical device may receive a first coating with the preferred poly[bis(trifluoroethoxy)phosphazene] polymer of the present invention, followed by an intermediate coating of nitric oxide-eluting or other smooth muscle relaxant compounds, followed by a second coating of the poly[bis(trifluoroethoxy)phosphazene] polymer as described herein. In such embodiments with a first and second coating of the poly[bis(trifluoroethoxy)phosphazene] polymer, the first and second coatings may each be bioabsorbable or non-bioabsorbable.

The medical devices disclosed herein may comprise the poly[bis(trifluoroethoxy)phosphazene] polymer represented by formula (I) in various forms: as a coating, as a film, or as a solid structural component. When used as a coating or film in embodiments of the present invention, the poly[bis(trifluoroethoxy)phosphazene] polymer may be provided in varying degrees of porosity, or as a solid surface. Such coatings may be achieved by spin coating, spray coating, meniscus coating, roller curtain and extrusion coating techniques, in addition to plasma deposition and electrophoretic photoresistance methods.

Similarly, the poly[bis(trifluoroethoxy)phosphazene] polymer may be provided as either a bioabsorbable or non-bioabsorbable form as most appropriate in various embodiments of the present invention. In various embodiments of the present invention, two or more coatings of the poly[bis(trifluoroethoxy)phosphazene] polymer may be applied to the surface of a medical device, and the two or more coatings of the poly[bis(trifluoroethoxy)phosphazene] polymer may be independently provided as bioabsorbable or non-bioabsorbable.

In one embodiment of the present invention an adhesion promoter may be provided in a layer between the surface of the substrate and the polymeric coating.

In exemplary embodiments of the present invention, the adhesion promoter is an organosilicon compound, preferably an amino-terminated silane or a compound based on an aminosilane, or an alkylphosphonic acid. Aminopropyltrimethoxysilane is a preferred adhesion promoter according to the present invention.

In various exemplary embodiments of the present invention, the adhesion promoter particularly improves the adhesion of the coating to the surface of the implant material through coupling of the adhesion promoter to the surface of the implant material, through, for instance, ionic and/or covalent bonds, and through further coupling of the adhesion promoter to reactive components, particularly to the antithrombogenic polymer of the coating, through, for instance, ionic and/or covalent bonds.

It will be appreciated by those possessing ordinary skill in the art that changes could be made to the embodiments described above without departing from the broad inventive concept thereof. It is understood, therefore, that this invention is not limited to the particular embodiments disclosed, but it is intended to cover modifications within the spirit and scope of the present invention as defined by the appended claims.

We claim:

1. A blood storage or administration device, comprising:
a. A container or administration device for the storage or administration of blood or blood products;
b. a specific polyphosphazene component, said polyphosphazene having the formula: 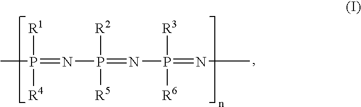
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof, and
c. a smooth muscle relaxant active agent.

2. The blood storage or handling device according to claim 1, wherein at least one of R¹ to R⁶ is an alkoxy group substituted with at least one fluorine atom.

3. The blood storage or handling device according to claim 1, wherein R¹ to R⁶ are selected independently from OCH₃, OCH₂CH₃, OCH₂CH₂CH₃, OCF₃ OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂, OCH₂CH═CH₂, OCH₂CH₂CH═CH₂ or any combination thereof.

4. The blood storage or handling device according to claim 1, wherein the polyphosphazene is poly[bis(2,2,2-trifluoroethoxy)]phosphazene or a derivative of poly[bis(2,2,2-trifluoroethoxy)]phosphazene.

5. The blood storage or handling device according to claim 1, wherein said polyphosphazene component is a coating for said container or administration device.

6. The blood storage or handling device according to claim 1, wherein said smooth muscle relaxant active agent is releasably bonded to said polyphosphazene component.

7. The blood storage or handling device according to claim 1, wherein said smooth muscle relaxant active agent is a compound capable of producing nitric oxide or other bioactive nitrogen compounds for release into said container or administration device.

8. The blood storage or handling device according to claim 1, wherein said container is a blood bag.

9. The blood storage or handling device according to claim 1, wherein said administration device is a blood administration tubing.

10. A coating for a blood storage or handling device, comprising:
a. a specific polyphosphazene coating, said polyphosphazene having the formula: 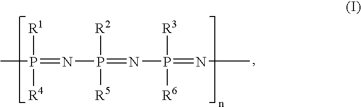
n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof.
b. a smooth muscle relaxant active agent.

11. The coating according to claim 10, wherein at least one of R¹ to R⁶ is an alkoxy group substituted with at least one fluorine atom.

12. The coating according to claim 10, wherein R¹ to R⁶ are selected independently from OCH₃, OCH₂CH₃, OCH₂CH₂CH₃, OCF₃, OCH₂CF₃, OCH₂CH₂CF₃, OCH₂CF₂CF₃, OCH(CF₃)₂, OCCH₃(CF₃)₂, OCH₂CF₂CF₂CF₃, OCH₂(CF₂)₃CF₃, OCH₂(CF₂)₄CF₃, OCH₂(CF₂)₅CF₃, OCH₂(CF₂)₆CF₃, OCH₂(CF₂)₇CF₃, OCH₂CF₂CHF₂, OCH₂CF₂CF₂CHF₂, OCH₂(CF₂)₃CHF₂, OCH₂(CF₂)₄CHF₂, OCH₂(CF₂)₅CHF₂, OCH₂(CF₂)₆CHF₂, OCH₂(CF₂)₇CHF₂ OCH₂CH═CH₂, OCH₂CH₂CH═CH₂, or any combination thereof.

13. The coating according to claim 10, wherein the polyphosphazene is poly[bis(2,2,2-trifluoroethoxy)]phosphazene or a derivative of poly[bis(2,2,2-trifluoroethoxy)]phosphazene.

14. The coating according to claim 10, wherein said smooth muscle relaxant active agent is releasably bonded to said polyphosphazene component.

15. The coating according to claim 10, wherein said smooth muscle relaxant active agent is a compound capable of producing nitric oxide or other bioactive nitrogen compounds for release within a blood storage or handling device.

16. The coating according to claim 10, wherein said polyphosphazene component is a coating for an interior surface of a blood bag.

17. The coating according to claim 10, wherein said polyphosphazene component is a coating for an interior surface of a blood administration tubing.

18. The coating according to claim 10, wherein said coating is applied to a substrate surface of a blood storage or handling device by dip coating, spray coating, spin coating, brush coating, electrostatic coating, electroplating, or electron beam-physical vapor deposition.

19. The coating according to claim 10, wherein said blood storage or handling device contains blood or blood products.

20. A method of enhancing the storage of blood or blood products and reducing the incidence of post-transfusion complications, comprising:
a. providing a blood storage or handling device further comprising
(i) a container or administration device for the storage or administration of blood or blood products;
(ii) a specific polyphosphazene component, said polyphosphazene having the formula: 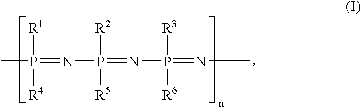
where n is 2 to ∞; and
R¹ to R⁶ are each selected independently from alkyl, aminoalkyl, haloalkyl, thioalkyl, thioaryl, alkoxy, haloalkoxy, aryloxy, haloaryloxy, alkylthiolate, arylthiolate, alkylsulphonyl, alkylamino, dialkylamino, heterocycloalkyl comprising one or more heteroatoms selected from nitrogen, oxygen, sulfur, phosphorus, or a combination thereof, or heteroaryl comprising one or more heteroatoms selected from nitrogen, oxygen, sulfur, phosphorus, or a combination thereof; and
(iii) a smooth muscle relaxant active agent;
b. storing blood within a blood storage or handling device as described herein,
c. administering blood or blood products to a patient using said blood storage or handling device; and
d. releasing said smooth muscle relaxant active agent.

21. The method according to claim 20, wherein the polyphosphazene is poly[bis(2,2,2-trifluoroethoxy)]phosphazene or a derivative of poly[bis(2,2,2-trifluoroethoxy)]phosphazene.

22. The method according to claim 20, wherein said polyphosphazene component is a coating for in inner surfaces of said blood storage or handling devices.

23. The method according to claim 20, wherein said smooth muscle relaxant active agent is releasably bonded to said polyphosphazene component.

24. The method according to claim 20, wherein said smooth muscle relaxant active agent is a compound capable of producing nitric oxide or other bioactive nitrogen compounds upon chemical release.

25. The method according to claim 20, further comprising activating or accelerating said release of smooth muscle relaxant active agent at a desired time by administering releasing agents or energy to said blood storage or handling devices.
